(19) United States
(12) Patent Application Publication
(10) Pub. No.: US 2025/0136760 A1
(43) Pub. Date: May 1, 2025

(54) AMINO-ACID- AND AMINO-ALCOHOL-BASED POLYAMINE COMPOUNDS AND MEDICAL HYDROGELS FORMED THEREFROM

(71) Applicant: Boston Scientific Scimed, Inc., Maple Grove, MN (US)

(72) Inventors: Nicolas Ball-Jones, Minneapolis, MN (US); Rick Dorn, Plymouth, MN (US); Cristian Parisi, Boston, MA (US); Yen-Hao Hsu, Shrewsbury, MA (US); Joseph Thomas Delaney, Jr., Minneapolis, MN (US); Sasha Viola Rios, Minneapolis, MN (US); Kyle A. Kalstabakken, Eagan, MN (US)

(73) Assignee: Boston Scientific Scimed, Inc., Maple Grove, MN (US)

(21) Appl. No.: 18/934,579

(22) Filed: Nov. 1, 2024

Related U.S. Application Data
(60) Provisional application No. 63/595,099, filed on Nov. 1, 2023.
(60) Provisional application No. 63/624,606, filed on Jan. 24, 2024.

(51) Int. Cl. C08G 73/02; A61K 9/06; A61K 31/785

(52) U.S. Cl. CPC C08G 73/02; A61K 9/06; A61K 31/785

(57) ABSTRACT

In some aspects, the present disclosure pertains to amino-acid-based polyamine compounds that are formed by a ring-opening reaction of two or more amino acid N-carboxyanhydride molecules with a polyol having two or more hydroxyl groups under acid catalyzed conditions. In some aspects, the present disclosure pertains to amino-acid-based polyamine compounds that are formed by ester coupling reaction of two or more amine-protected amino acid molecules with a polyol having two or more hydroxyl groups in the presence of an ester coupling agent. In some aspects, the present disclosure pertains to amino-alcohol-based polyamine compounds that are formed by an ester coupling reaction of two or more amine-protected amino alcohol molecules having one or more amino groups and a single hydroxyl group with a polycarboxylic acid compound having two or more carboxyl groups in the presence of an ester coupling agent.

### CROSS-REFERENCE TO RELATED APPLICATIONS

This application claims the benefit of U.S. Provisional Patent Application Ser. No. 63/595,099 filed on Nov. 1, 2023, and U.S. Provisional Patent Application Ser. No. 63/624,606 filed on Jan. 24, 2024, the disclosures of which are incorporated herein by reference.

### FIELD

The present disclosure relates to amino-acid- and amino-alcohol based polyamine compounds, to the use of such compounds as crosslinking agents for forming hydrogels, and to hydrogels formed from such compounds. The crosslinking agents and hydrogels are useful, for example, in various medical applications

### BACKGROUND

SpaceOAR® is based on a multi-arm polyethylene glycol (PEG) polymer functionalized with succinimidyl glutarate as activated end groups that, above a specific pH, rapidly crosslink with lysine trimer (Lys-Lys-Lys) in vivo to form a hydrogel. This product has become a very successful, clinically-used biomaterial in prostate cancer therapy. A further improvement based on this structure is that a portion the succinimidyl glutarate end groups have been functionalized with 2,3,5-triiiodobenzamide groups, thereby providing radiopacity. This hydrogel, known by the trade name of SpaceOAR® Vue, is the radiopaque version of SpaceOAR® for prostate medical applications.

While the current synthetic route for forming the trilysine crosslinker is sufficient in that it produces a monodisperse, well-defined small molecule oligomer, the process is tedious as it involves five steps and includes multiple protection/deprotection reactions.

For these and other reasons, alternative crosslinking agents are desired for forming hydrogels and for forming crosslinked hydrogels that provide enhanced radiopacity.

### SUMMARY

In some aspects, the present disclose provides amino-acid-based polyamine compounds that are formed by ring-opening reaction of (a) two or more amino acid N-carboxyanhydride molecules with (b) a polyol having two or more hydroxyl groups under acid catalyzed conditions.

In some embodiments, the amino acid N-carboxyanhydride molecules are selected from glycine N-carboxyanhydride, lysine N-carboxyanhydride, ornithine N-carboxyanhydride, and cysteine N-carboxyanhydride.

In some aspects, the present disclose provides amino-acid-based polyamine compounds that are formed by an ester coupling reaction of (a) two or more amine-protected amino acid molecules with (b) a polyol having two or more hydroxyl groups in the presence of an ester coupling agent.

In some embodiments, the amine-protected amino acid is selected from amine-protected glycine, amine-protected lysine, amine-protected ornithine, and amine-protected cysteine.

In some aspects, the present disclose provides amino-acid-based polyamine compounds that comprises (a) a residue of a polyol having two or more hydroxyl groups and (b) two or more single amine-terminated amino acid residues that are each linked by an ester bond at a site of a residue of one of the two or more hydroxyl groups.

In some embodiments, the amine-terminated amino acid residues are selected from amine-terminated glycine residues, amine-terminated lysine residues, amine-terminated ornithine residues, and amine-terminated cysteine residues.

In some embodiments, which can be used with any of the above aspects and embodiments, the polyol has between 3 and 20 hydroxyl groups.

In some aspects, the present disclosure pertains to amino-alcohol-based polyamine compounds that are formed by an ester coupling reaction of two or more amine-protected amino alcohol molecules having one or more amino groups and a single hydroxyl group with (b) a polycarboxylic acid compound having two or more carboxyl groups, preferably in the presence of an ester coupling agent.

In some embodiments, the amine-protected amino alcohol is an amine-protected C₂-C₁₀-amino alcohol having one, two, three or four amino groups and a single hydroxyl group.

In some aspects, the present disclosure pertains to amino-alcohol-based polyamine compounds that comprises (a) a residue of a polycarboxylic acid compound having two or more carboxyl groups and (b) two or more single amine-terminated amino alcohol residues that each have one or more amino groups and each are linked by an ester bond at a site of a residue of one of the two or more carboxyl groups.

In some embodiments, the amine-terminated amino alcohol residues are amine-terminated C₂-C₁₀-amino alcohol residues having one, two, three or four amino groups.

In some embodiments, which can be used with the above aspects and embodiments, the polycarboxylic acid has between 3 and 20 carboxyl groups.

In other aspects, the present disclosure pertains to systems for forming a hydrogel that comprises the amino-acid- or amino-alcohol-based polyamine compound of any of the above aspects and embodiments and a polymer that that forms crosslinks with the amino-acid- or amino-alcohol-based polyamine compound.

In some embodiments, the polymer that forms crosslinks with the amino-acid- or amino-alcohol-based polyamine compound is a reactive multi-arm polymer that comprises a plurality of hydrophilic polymer arms comprising hydrophilic polymer segments and reactive end groups that covalently crosslink with primary amine groups of the amino-acid- or amino-alcohol-based polyamine compound. In some of these embodiments, the hydrophilic polymer segments are selected from polyalkylene oxide segments, polyester segments, polyoxazoline segments, polydioxanone segments, and polypeptide segments. In some of these embodiments, the reactive groups are electrophilic groups selected from imidazole esters, imidazole carboxylates, benzotriazole esters, or imide esters. In some of these embodiments, the reactive groups are cyclic imide ester groups.

In some of these embodiments, the polymer that forms crosslinks with the amino-acid- or amino-alcohol-based polyamine compound is an anionic polymer that comprises a plurality of anionic groups that ionically crosslink with the plurality of primary amine groups of the amino-acid- or amino-alcohol-based polyamine compound. In some of these embodiments, the anionic polymer comprises a plurality of carboxyl groups, sulfonate groups, sulfate groups, phosphate groups, or phosphonate groups.

In some aspects, the present disclosure pertains to medical hydrogels that formed by crosslinking the amino-acid- or amino-alcohol-based polyamine compound and the polymer that forms crosslinks with the amino-acid- or amino-alcohol-based polyamine compound of the system of any of the above aspects and embodiments.

In some aspects, the present disclosure pertains to methods of treatment comprising administering to a subject a mixture that comprises the amino-acid- or amino-alcohol-based polyamine compound of any of the above aspects and embodiments and a polymer that forms crosslinks with the amino-acid- or amino-alcohol-based polyamine compound under conditions such that the amino-acid-based or amino-alcohol-based polyamine compound and the polymer crosslink after administration.

In some aspects, the present disclosure pertains to methods of forming an amino-acid-based polyamine compound comprising performing a ring-opening reaction of two or more amino acid N-carboxyanhydride molecules with a polyol having two or more hydroxyl groups under acid catalyzed conditions.

In some embodiments, the amino acid N-carboxyanhydride molecules are protected amino acid N-carboxyanhydride molecules and the method further comprises a deprotection step after preforming the ring-opening reaction.

In some embodiments, which can be used with the above aspects and embodiments, the reaction mixture comprises an acid catalyst selected from HCl, phosphoric acid, p-toluene sulfonic acid, methane sulfonic acid, and sulfuric acid.

In some embodiments, which can be used with the above aspects and embodiments, the ring-opening reaction is performed with a molar excess of amino acid N-carboxyanhydride molecules relative to the number hydroxyl groups in the reaction mixture.

In some embodiments, which can be used with the above aspects and embodiments, the ring-opening reaction is performed with a molar excess of the hydroxyl groups relative to the number of amino acid N-carboxyanhydride molecules in the reaction mixture.

In some aspects, the present disclosure pertains to methods of forming an amino-acid-based polyamine compound comprising performing an ester coupling reaction of two or more amine-protected amino acid molecules with a polyol having two or more hydroxyl groups in the present of an ester-coupling agent; and performing a deprotection step after preforming the ester coupling reaction.

In some embodiments, the ester-coupling agent is a carbodiimide coupling agent compound.

In some embodiments, which can be used with the above aspects and embodiments, the ester coupling reaction is performed with a molar excess of amine-protected amino acid molecules relative to the number hydroxyl groups in the reaction mixture.

In some embodiments, which can be used with the above aspects and embodiments, the ester coupling reaction is performed with a molar excess of the hydroxyl groups relative to the number of amine-protected amino acid molecules in the reaction mixture.

In some aspects, the present disclosure pertains to methods of forming an amino-alcohol-based polyamine compound comprising performing an ester coupling reaction of two or more amine-protected amino alcohol molecules with a polycarboxylic acid molecule having two or more carboxyl groups in the present of an ester-coupling agent and performing a deprotection step after preforming the ester coupling reaction.

In some embodiments, the ester-coupling agent is a carbodiimide coupling agent.

In some embodiments, which can be used with the above aspects and embodiments, the ester coupling reaction is performed with a molar excess of amine-protected amino alcohol molecules relative to the number carboxyl groups in the reaction mixture.

In some embodiments, which can be used with the above aspects and embodiments, the ester coupling reaction is performed with a molar excess of the carboxyl groups relative to the number of amine-protected amino alcohol molecules in the reaction mixture.

The above and other aspects, embodiments, features and benefits of the present disclosure will be readily apparent from the following detailed description.

### BRIEF DESCRIPTION OF THE DRAWINGS

FIG. 1 schematically illustrates a process for forming an amino-acid-based polyamine compound from a polyol and an amino acid N-carboxyanhydride, in accordance with an embodiment of the present disclosure.

FIG. 2 schematically illustrates a process for forming an amino-acid-based polyamine compound from glycerol and glycine N-carboxyanhydride, in accordance with an embodiment of the present disclosure.

FIG. 3 schematically illustrates a process for forming an amino-acid-based polyamine compound from myo-inositol and glycine N-carboxyanhydride, in accordance with an embodiment of the present disclosure.

FIG. 4 schematically illustrates a process for forming an amino-acid-based polyamine compound from glycerol and amine-protected glycine, in accordance with an embodiment of the present disclosure.

FIG. 5 schematically illustrates a process for forming an amino-acid-based polyamine compound from iodixanol and amine-protected glycine, in accordance with an embodiment of the present disclosure.

FIG. 6 schematically illustrates a process for forming an amino-alcohol-based polyamine compound from N,N,N′,N′-tetrakis(carboxymethyl)-ethane-1,2-diamine and amine-protected ethanolamine, in accordance with an embodiment of the present disclosure.

FIG. 7 schematically illustrates a process for forming an amino-alcohol-based polyamine compound from 2,4,6-triiodobenzene-1,3,5-tricarboxylic acid and amine-protected ethanolamine, in accordance with an embodiment of the present disclosure.

FIG. 8 schematically illustrates a process for forming an amino-alcohol-based polyamine compound from 2,4,6-triiodobenzene-1,3,5-tricarboxylic acid and amine-protected 2-[Bis(2-aminoethyl)amino]ethanol, in accordance with a further embodiment of the present disclosure.

FIG. 9 schematically illustrates a delivery device, in accordance with an embodiment of the present disclosure.

### DETAILED DESCRIPTION

In some aspects, the present disclosure provides amino-acid-based polyamine compounds that are formed by (a) ring-opening reaction of amino acid N-carboxyanhydrides (NCAs) with polyols having two or more hydroxyl groups or (b) ester coupling reaction of amine-protected amino acids with polyols having two or more hydroxyl groups.

Amino-acid-based polyamine compounds in accordance with the present disclosure comprise a polyol residue and at least two amino acid residues which are each covalently linked to the polyol residue through an ester group.

In embodiments where the amino-acid-based polyamine compound is formed by a ring-opening reaction of an NCA with a polyol having two or more hydroxyl groups, the amino acid resides comprise a primary amine group that arises from the ring opening process and a side group that is specific to each NCA.

In embodiments where the amino-acid-based polyamine compound is formed by an ester coupling reaction of an amine-protected amino acid with a polyol having two or more hydroxyl groups, the amino acid resides likewise comprise a primary amine group (after deprotection of the alpha-amine group) and a side group that is specific to each amino acid.

With regard to the ring-opening route, by performing the ring-opening reaction under acid catalyzed conditions, a single amino acid NCA is reacted with a single hydroxyl group of the polyol, without triggering (or desiring) further NCA polymerization. Consequently, at most one amino acid residue is attached at the site of each hydroxyl group of the polyol. In this regard, by performing the reaction with a molar excess of amino acid NCAs relative to the total number of moles of hydroxyl groups provided by the polyol in the reaction mixture, a single amino acid NCA will be reacted with each of the hydroxyl groups of the polyol, and no hydroxyl groups will remain in the resulting amino-acid-based polyamine compound. On the other hand, by performing the reaction with a molar excess of hydroxyl groups relative to the amino acid NCAs, some of the hydroxyl groups of the of the polyol will remain unreacted in the resulting amino-acid-based polyamine compound.

Acid catalysts that can be used on conjunction with the ring opening reactions of the present disclosure include HCl, phosphoric acid, p-toluene sulfonic acid, methane sulfonic acid, and sulfuric acid, among others.

Amino acid N-carboxyanhydrides (NCAs) for use in the present disclosure include those of the formula,

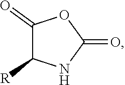

where R is a side group that is specific to each amino acid NCA. Amino acid NCAs include both canonical and non-canonical NCAs. Particular examples of R groups are as follows: alanine (R=—CH₃), cysteine (R=—CH₂SH), aspartic acid (R=—CH₂COOH), glutamic acid (R=—CH₂CH₂COOH), phenylalanine (R=—CH₂C₆H₅), glycine (R=—H), histidine

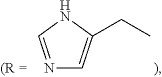

isoleucine (R=—CH(CH₃)CH₂CH₃), lysine (R=—(CH₂)₄NH₂), ornithine (R=—(CH₂)₃NH₂), leucine (R=—CH₂CH(CH₃)₂), methionine (R=—CH₂CH₂SCH₃), asparagine (R=—CH₂CONH₂), glutamine (R=—CH₂CH₂CONH₂), arginine (R=—(CH₂)₃NH—C(NH)NH₂), serine (R=—CH₂OH), threonine (R=—CH(OH)CH₃), selenocysteine (R=—CH₂SeH), valine (R=—CH(CH₃)₂), tryptophan

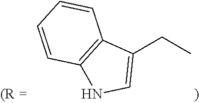

and tyrosine (R=—CH₂—C₆H₄OH), among others.

Regardless of the particular amino acid NCA that is selected, any amino acid residue that is appended to the polyol will have a free amine group at the end of the process.

Depending on the nature of the R group of the amino acid NCA, it may be desirable to employ protective groups to prevent unwanted side reactions from occurring during the ring opening process. For example, it may be desirable to protect amine, carboxylic acid, alcohol, or thiol groups during the ring opening process. Examples of protective groups for use in conjunction with the present disclosure include tert-butoxycarbonyl (Boc) groups, carboxybenzyl (Cbz) or (Z) groups, trifluoroacetyl (TFA) groups, 6-nitroveratryloxycarbonyl (Nvoc) groups, 9-fluorenylmethoxycarbonyl (Fmoc) groups, allyloxycarbonyl (Alloc) groups, trityl (Trt) groups, t-butyl (t-Bu) groups, benzyl ester (β-benzyl) groups, O-benzyl groups, S-ethylsulfonyl groups, 2,4-dimethoxybenzyl (Dmb), tert-butyldimethylsilyl (TBDMS), allyl, ortho-nitrobenzyl (PNB), para-methylbenzyl (Meb), and acetamidomethyl (Acm) groups, among others.

In some embodiments, amino acid NCAs are employed that contain protected amine side groups. For example, amino acid NCAs may be employed that contain protected primary amine side groups such as aminoalkyl groups (e.g., C₁-C₆-aminoalkyl groups, including aminomethyl, 2-aminoethyl, 3-aminopropyl, 4-aminobutyl, 5-aminopentyl and 6-aminohexyl groups, as well as isomers of the same) and protected imidazolyl side groups. Particular examples include amine-protected lysine NCA monomers (where the protected group is a 4-aminobutyl group), amine-protected ornithine NCA monomers (where the protected group is a 3-aminopropyl group), and amine-protected histidine NCA monomers (where the protected group is an imidazolyl group), among others. More particular examples of such amine-protected amino acid NCAs include lysine (Boc)-NCA,

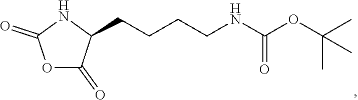 [SMILES: CC(C)(C)OC(=O)NCCCC[C@@H]1NC(=O)OC1=O]

ornithine (Boc)-NCA,

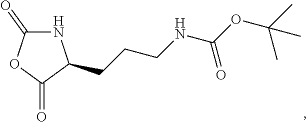 [SMILES: CC(C)(C)OC(=O)NCCC[C@@H]1NC(=O)OC1=O]

lysine (Z)-NCA, also known as Lysine (Cbz)-NCA,

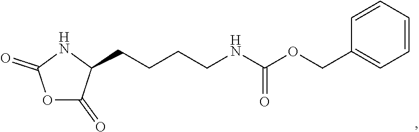 [SMILES: O=C(NCCCC[C@@H]1NC(=O)OC1=O)OCc1ccccc1]

ornithine (Z)-NCA, also known as ornithine (Cbz)-NCA,

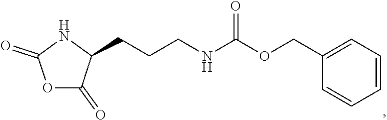 [SMILES: O=C(NCCC[C@@H]1NC(=O)OC1=O)OCc1ccccc1]

and lysine (TFA)-NCA,

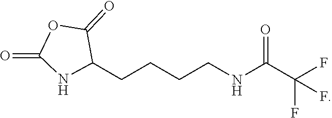 [SMILES: O=C1NC(CCCCNC(=O)C(F)(F)F)C(=O)O1]

N(Im)-(2,4-dinitrophenyl)-L-histidine NCA.

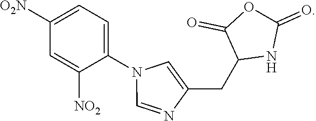 [SMILES: O=C1NC(Cc2cn(-c3ccc([N+](=O)[O-])cc3[N+](=O)[O-])cn2)C(=O)O1]

Additional examples of amino acid NCAs include protected amino acid NCAs that contain protected carboxyl side groups (e.g., C₁-C₆-carboxyalkyl groups, including carboxymethyl, 2-carboxyethyl, 3-carboxypropyl, 4-carboxybutyl, 5-carboxypentyl and 6-carboxyhexyl groups, as well as isomers of the same) such as β-benzyl L-Aspartic acid NCA,

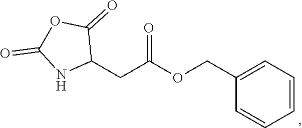 [SMILES: O=C(CC1NC(=O)OC1=O)OCc1ccccc1]

and γ-benzyl-L-glutamic acid NCA,

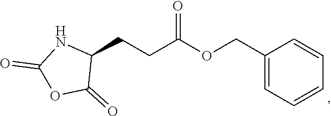 [SMILES: O=C(CC[C@@H]1NC(=O)OC1=O)OCc1ccccc1]

amino acid NCAs with protected hydroxyl side groups (e.g., C₁-C₆-hydroxyalkyl groups, including hydroxymethyl, 2-hydroxyethyl, 3-hydroxypropyl, 4-hydroxybutyl, 5-hydroxypentyl and 6-hydroxyhexyl groups, as well as isomers of the same, and phenol side groups) such as O-benzyl-L-serine NCA,

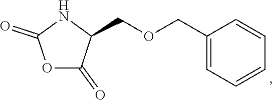 [SMILES: O=C1N[C@@H](COCc2ccccc2)C(=O)O1]

### and O-benzyl-L-tyrosine NCA

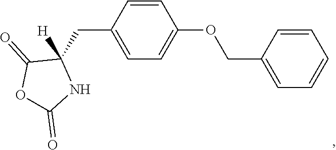 [SMILES: O=C1N[C@@H](Cc2ccc(OCc3ccccc3)cc2)C(=O)O1]

and protected amino acid NCAs with protected thiol side groups (e.g., C₁-C₆-thiolalkyl groups, including thiolmethyl, 2-thiolethyl, 3-thiolpropyl, 4-thiolbutyl, 5-thiolpentyl and 6-thiolhexyl groups, as well as isomers of the same) such as S-tert-butylmercapto cysteine NCA,

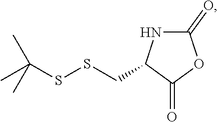

among others.

Subsequent to performing the acid-catalyzed ring opening process, the resulting protected amino acid resides can be deprotected using known techniques.

Turning now to the ester coupling route, by performing the reaction with a molar excess of amine-protected amino acids relative to the total number of moles of hydroxyl groups provided by the polyol in the reaction mixture, a single amino acid will be reacted with each of the hydroxyl groups of the polyol, and no hydroxyl groups will remain in the resulting amino-acid-based polyamine compound. On the other hand, by performing the reaction with a molar excess of hydroxyl groups relative to amine-protected amino acids, some of the hydroxyl groups of the of the polyol will remain unreacted in the resulting amino-acid-based polyamine compound.

The ester coupling between the carboxylic acid and hydroxyl groups may be performed in the presence of a suitable coupling agent, for example, a carbodiimide coupling agent, such as N,N′-dicyclohexylcarbodiimide (DCC), 1-ethyl-3-(3-dimethylaminopropyl) carbodiimide (EDC), or N,N′-diisopropylcarbodiimide (DIC), among others. The ester linkage that is formed is hydrolysable.

Amine-protected amino acids for use herein include both canonical and non-canonical amine-protected amino acids.

Protective groups for the alpha-amine group of the amine-protected amino acids include tert-butoxycarbonyl (Boc) groups, 9-fluorenylmethoxycarbonyl (Fmoc) groups, tert-butyl (t-Bu) groups, and benzyl (Bn) groups. In the case where a Boc protective group is employed, amine-protected amino acids for use in the present disclosure include those of the formula,

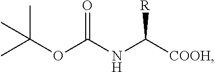

where R is a side group that is specific to each amino acid that is protected. In the case where an Fmoc protective group is employed, amine-protected amino acids for use in the present disclosure include those of the formula,

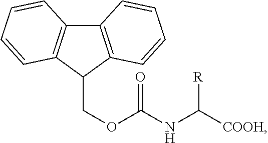

where R is a side group that is specific to each amino acid that is protected. As above, examples of R groups are as follows: glycine (R=—H), alanine (R=—CH₃), cysteine (R=—CH₂SH), aspartic acid (R=—CH₂COOH), glutamic acid (R=—CH₂CH₂COOH), phenylalanine (R=—CH₂C₆H₅), histidine

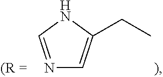

isoleucine (R=—CH(CH₃)CH₂CH₃), lysine (R=—(CH₂)₄NH₂), ornithine (R=—(CH₂)₃NH₂), leucine (R=—CH₂CH(CH₃)₂), methionine (R=—CH₂CH₂SCH₃), asparagine (R=—CH₂CONH₂), glutamine (R=—CH₂CH₂CONH₂), arginine (R=—(CH₂)₃NH—C(NH)NH₂), serine (R=—CH₂OH), threonine (R=—CH(OH)CH₃), selenocysteine (R=—CH₂SeH), valine (R=—CH(CH₃)₂), tryptophan

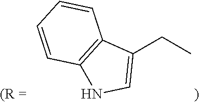

and tyrosine (R=—CH₂—C₆H₄OH), among others.

Regardless of the particular amino acid that is selected, any amino acid residue that is appended to the polyol will have at least one free amine group at the end of the deprotection process.

Depending on the nature of the R group of the amino acid, it may be desirable to employ additional protective groups to prevent unwanted R reactions from occurring during the ester coupling process. For example, it may be desirable to protect non-alpha-amino R groups such as aminoalkyl groups (e.g., C₁-C₆-aminoalkyl groups, including aminomethyl, 2-aminoethyl, 3-aminopropyl, 4-aminobutyl, 5-aminopentyl and 6-aminohexyl groups, as well as isomers of the same) (e.g., for lysine, ornithine, etc.), carboxylic acid R groups such as carboxyalkyl groups (e.g., C₁-C₆-carboxyalkyl groups, including carboxymethyl, 2-carboxyethyl, 3-carboxypropyl, 4-carboxybutyl, 5-carboxypentyl and 6-carboxyhexyl groups, as well as isomers of the same) (e.g., for aspartic acid, glutamic acid, etc.), hydroxyl (alcohol) R groups (e.g., for serine, threonine, hydroxyproline, tyrosine), thiol R groups (e.g., for cysteine), guanidinium groups (e.g., for arginine), or imidazole groups (e.g., for histidine) during the ester coupling process.

Examples of additional protective groups for use in conjunction with the present disclosure include protective groups for amine R groups, such as tert-butoxycarbonyl (Boc) groups, carboxybenzyl (Cbz) groups, allyloxycarbonyl (Alloc) groups, or 6-nitroveratryloxycarbonyl (Nvoc), protective groups for carboxyl R groups, such as trityl (Trt), t-butyl (t-Bu), benzyl (Bn), 9-fluorenylmethyl (Fm), or 2,4-dimethoxybenzyl (Dmb) groups, protective groups for hydroxyl R groups, such as tert-butyldimethylsilyl (TBDMS), allyl, or o-nitrobenzyl (ONB), protective groups for guanidinium R groups such as 2,2,4,6,7-pentamethyldihydrobenzofuran-5-sulfonyl residue (Pbf) groups, protective groups for imidiazole R groups such as tosyl (Tos) or benzyloxymethyl (Bom) groups, protective groups for thiol R groups such as para-methylbenzyl (Meb), acetamidomethyl (Acm) and trityl (Trt) groups, among others. In the particular case of protected amino R groups, commercially available compounds include protected lysine, e.g., Boc-Lys (Boc)-OH,

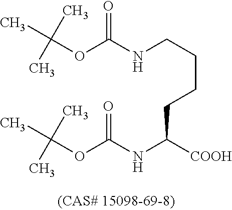 [SMILES: CC(C)(C)OC(=O)NCCCC[C@H](NC(=O)OC(C)(C)C)C(=O)O]

and protected ornithine, e.g., Boc-Orn(Boc)-OH,

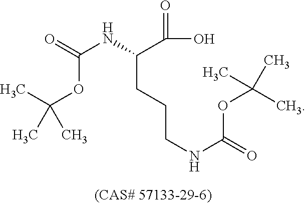 [SMILES: CC(C)(C)OC(=O)NCCC[C@H](NC(=O)OC(C)(C)C)C(=O)O]

Subsequent to performing the ester coupling process, the resulting protected amino acid resides can be deprotected using known techniques.

Polyols that can be used in conjunction with the ring-opening and ester coupling reactions described herein include those having two more hydroxyl groups, for example, containing anywhere from 2 to 100 hydroxyl groups (e.g., having 2, 3, 4, 5, 6, 7, 8, 9, 10, 12, 15, 20, 25, 30, 40, 50, 60, 70, 80, 90 or 100 hydroxyl groups). General classes of molecules including sugars (monosaccharides, disaccharides, trisaccharides, etc.), sugar alcohols, calixaranes, polyhedral oligomeric silsesquioxanes (POSS), cyclodextrin, polyhydroxylated polymers, catechins, flavanols, anthocyanins, stilbenes, and polyphenols, among others In general, the present disclosure pertains to alcohols that react with amino acid NCAs when initiated by acid catalysts, including primary alcohols, secondary alcohols, tertiary alcohols, and phenols.

Illustrative polyols include straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, short polymers (including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 2 to 100 monomer units in length.

Particular examples of polyols include glycerol,

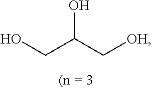

where n is the n is the number of —OH functional groups in the molecule), 1,2,4-butanetriol,

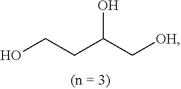

resveratrol,

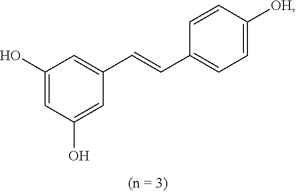 [SMILES: Oc1ccc(/C=C/c2cc(O)cc(O)c2)cc1]

3-(2-hydroxyethyl)pentane-1,5-diol,

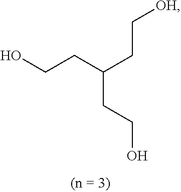

triethanolamine,

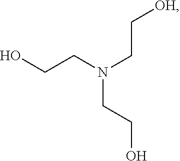

myo-inositol,

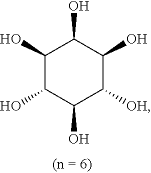 [SMILES: O[C@H]1[C@H](O)[C@@H](O)[C@H](O)[C@@H](O)[C@H]1O]

sorbitol,

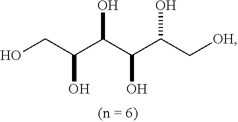 [SMILES: OC[C@@H](O)[C@@H](O)[C@H](O)[C@@H](O)CO]

tripentaerythritol,

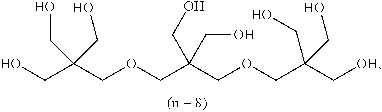 [SMILES: OCC(CO)(CO)COCC(CO)(CO)COCC(CO)(CO)CO]

epicatechin

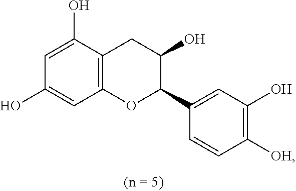 [SMILES: Oc1cc(O)c2c(c1)O[C@H](c1ccc(O)c(O)c1)[C@H](O)C2]

epicatechin gallate,

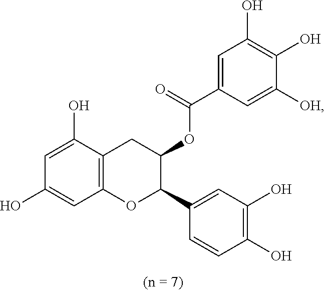 [SMILES: O=C(O[C@@H]1Cc2c(O)cc(O)cc2O[C@@H]1c1ccc(O)c(O)c1)c1cc(O)c(O)c(O)c1]

maltohexaose,

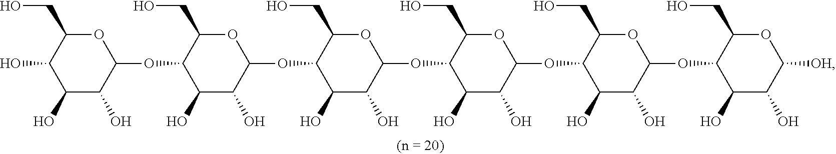 [SMILES: OC[C@H]1OC(O[C@H]2[C@H](O)[C@@H](O)C(O[C@H]3[C@H](O)[C@@H](O)C(O[C@H]4[C@H](O)[C@@H](O)C(O[C@H]5[C@H](O)[C@@H](O)C(O[C@H]6[C@H](O)[C@@H](O)[C@@H](O)O[C@@H]6CO)O[C@@H]5CO)O[C@@H]4CO)O[C@@H]3CO)O[C@@H]2CO)[C@H](O)[C@@H](O)[C@@H]1O]

and cyclodextrins having 4-10 glucose units (n=12-30), among many others.

In some embodiments, iodinated polyols may be employed to provide the resulting polyamine compound with radiopacity. In some of these embodiments, the iodinated polyols are compounds that comprise 2 or more hydroxyl groups, and one or more iodinated aromatic groups.

Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with the two or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, amine groups, ether groups, ester groups, or carbonate groups, among others.

Specific examples of iodinated polyols for use in the present disclosure include known iodinated contrast agents, whose biocompatibility has been demonstrated to be reasonably well tolerated. Illustrative iodinated polyols include 2,4,6-triiodobenzene-1,3,5-triol,

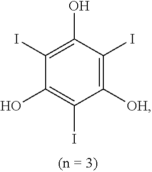 [SMILES: Oc1c(I)c(O)c(I)c(O)c1I]

iopromide,

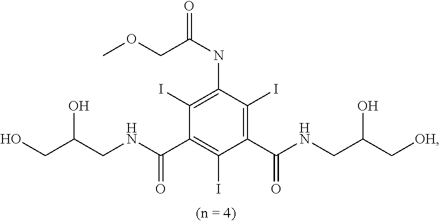 [SMILES: COCC(=O)Nc1c(I)c(C(=O)NCC(O)CO)c(I)c(C(=O)NCC(O)CO)c1I]

iopamidol,

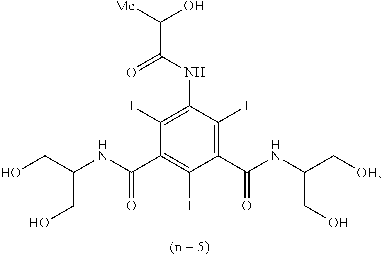 [SMILES: CC(O)C(=O)Nc1c(I)c(C(=O)NC(CO)CO)c(I)c(C(=O)NC(CO)CO)c1I]

iohexol,

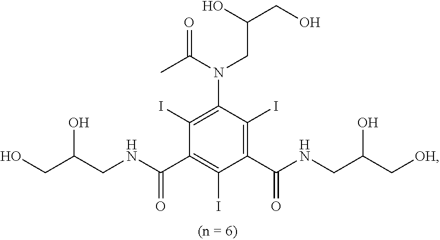 [SMILES: CC(=O)N(CC(O)CO)c1c(I)c(C(=O)NCC(O)CO)c(I)c(C(=O)NCC(O)CO)c1I]

ioversol,

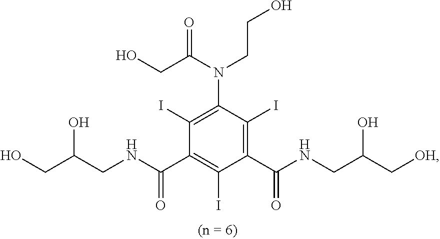 [SMILES: O=C(NCC(O)CO)c1c(I)c(C(=O)NCC(O)CO)c(I)c(N(CCO)C(=O)CO)c1I]

and iodixanol,

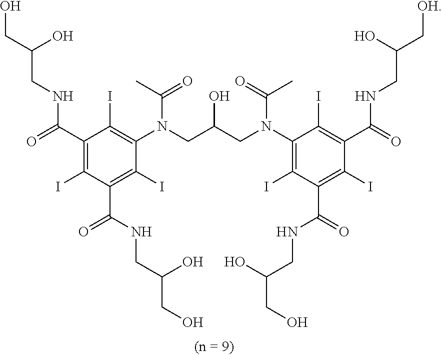 [SMILES: CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

In other embodiments, radiopacity can be introduced into the polyamine compounds by using iodinated amino acid NCA derivatives in the ring-opening synthesis or amine-protected iodinated amino acids in the ester coupling reaction. For example, iodinated phenylalanine NCA (e.g., 4-iodo-L-phenylalanine NCA), iodinated tyrosine NCA (e.g., 3-iodo-L-tyrosine NCA) may be used in the ring-opening synthesis in some embodiments, and amine-protected iodinated phenylalanine (e.g., protected 4-iodo-L-phenylalanine) or protected iodinated tyrosine (e.g., protected 3-iodo-L-tyrosine) may be used in the ester coupling reaction in some embodiments.

Notably, employing NCAs and amine-protected amino acids that contain protected amine side groups, such as those based on lysine or ornithine, effectively double the number of amine functional groups that can be appended to any given molecule. For instance, molecules such as those shown above that have an n=3 will be able to have six amine functional groups appended. This means that the maximum number of amine functional groups, m, that can be appended to a given polyol ranges from m=n (where an amino acid that does not contain a protected amine pendant group is employed) to m=2n (where an amino acid that contains a protected amine pendant group is employed). As previously noted, less than the maximum number of amine functional groups can be produced, for example, in the event that less than a molar excess of amino acid relative to the hydroxyl groups is employed. This is reflected in following table.

| | | | Possible # of |
|---|---|---|---|
| Compound | CAS# | —OH Groups | —NH₂ groups |
| Diethylene glycol | 111-46-6 | 2 | 2-4 |
| Ethylene glycol | 107-21-1 | 2 | 2-4 |
| Isosorbide | 652-67-5 | 2 | 2-4 |
| Neopentyl glycol | 126-30-7 | 2 | 2-4 |
| Triethylene glycol | 112-27-6 | 2 | 2-4 |
| 2-(Hydroxymethyl)-1,3- | 4704-94-3 | 3 | 2-6 |
| propanediol | | | |
| Glycerol | 56-81-5 | 3 | 2-6 |
| Trimethylolethane | 77-85-0 | 3 | 2-6 |
| Trimethylolpropane | 77-99-6 | 3 | 2-6 |
| 2,4,6-Triiodobenzene- | 57730-42-4 | 3 | 2-6 |
| 1,3,5-triol | | | |
| Diglycerol | 627-82-7 | 4 | 2-8 |
| Erythritol | 149-32-6 | 4 | 2-8 |
| Pentaerythritol | 115-77-5 | 4 | 2-8 |
| Iopromide | 73334-07-3 | 4 | 2-8 |
| Arabitol | 7643-75-6 | 5 | 2-10 |
| Triglycerol | 20411-31-8 | 5 | 2-10 |
| Xylitol | 87-99-0 | 5 | 2-10 |
| Iopamidol | 60166-93-0 | 5 | 2-10 |
| Dipentaerythritol | 126-58-9 | 6 | 2-12 |
| Mannitol | 69-65-8 | 6 | 2-12 |
| Sorbitol | 50-70-4 | 6 | 2-12 |
| Iohexol | 66108-95-0 | 6 | 2-12 |
| Ioversol | 87771-40-2 | 6 | 2-12 |
| Epicatechin gallate | 1257-08-5 | 7 | 2-14 |
| Octa-(3-hydroxypropyl) | 229967-81-1 | 8 | 2-16 |
| POSS | | | |
| Tripentaerythritol | 78-24-0 | 8 | 2-16 |
| Iodixanol | 92339-11-2 | 9 | 2-18 |
| Isomalt | 64519-82-0 | 9 | 2-18 |
| Maltitol | 585-88-6 | 9 | 2-18 |
| Maltohexose | 34620-77-4 | 20 | 2-40 |

Various specific ring-opening embodiments and ester coupling embodiments will now be discussed.

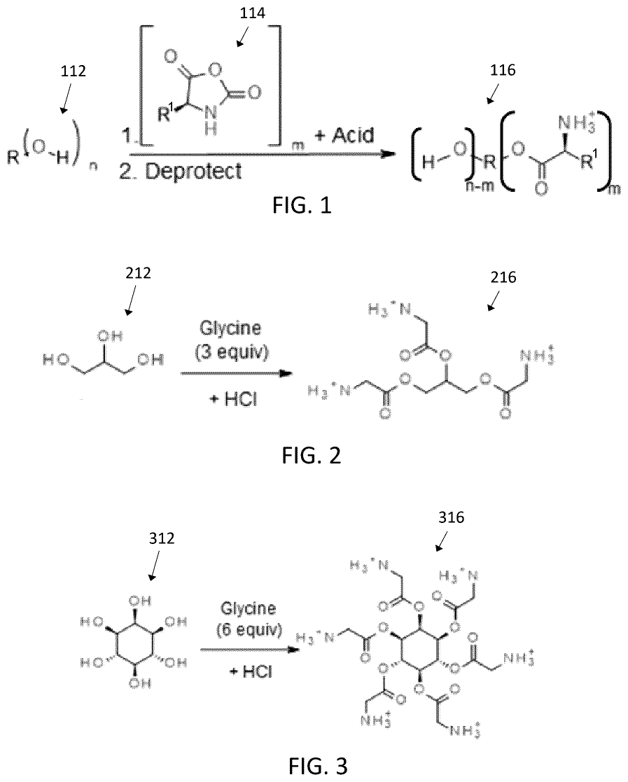

With reference now to FIG. 1, a general procedure is shown whereby a polyol (112) having n hydroxyl groups (where R is an organic group representing a remainder of the polyol) is reacted with m amino acid NCA molecules (114) (where R¹ represents a canonical or non-canonical amine group protected by a protecting group such as Cbz, Boc, TFA, Nvoc, or FMOC), followed by deprotection of the protecting group, to form a polyamine compound (116) having m appended amino acid residues and n-m unreacted hydroxyl groups.

With reference now to FIG. 2, a specific procedure is shown whereby 1 equivalent of glycerol (212) having 3 hydroxyl groups is reacted with 3 equivalents of glycine NCA to form a polyamine compound (216) having 3 appended glycine residues that are bonded to a residue of the glycerol through ester bonds.

Similarly, with reference to FIG. 3, another specific procedure is shown whereby 1 equivalent of myo-inositol (312) having 6 hydroxyl groups is reacted with 6 equivalents of glycine NCA to from a polyamine compound (316) having 6 appended glycine residues that are bonded to a residue of the myo-inositol through ester bonds.

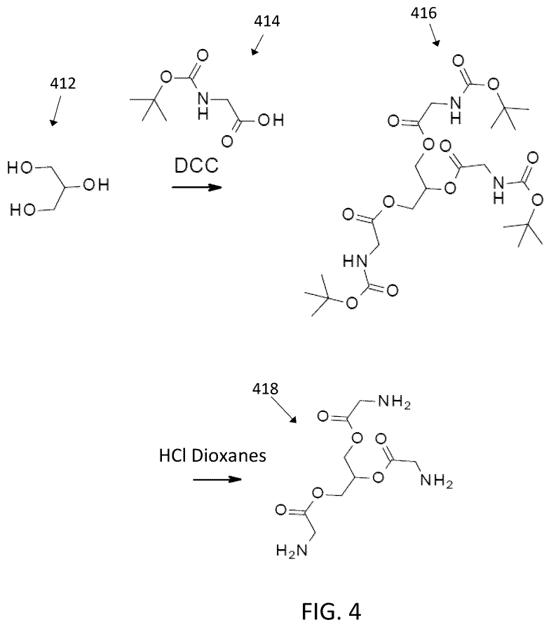

As an example of the ester coupling strategy and with reference to FIG. 4, glycerol (412) is allowed to react with an excess of Boc-glycine (414) in the presence of a carbodiimide coupling agent such as DCC, yielding 2,3-bis[[2-(tert-butoxycarbonylamino)acetyl]oxy]propyl 2-(tert-butoxycarbonylamino)acetate (416). This intermediate is then treated with a deprotecting agent, such as HCl in dioxanes, yielding 2,3-bis[(2-aminoacetyl)oxy]propyl 2-aminoacetate (418), which is a polyamine compound having 3 appended glycine residues that are bonded to a residue of the glycerol through ester bonds.

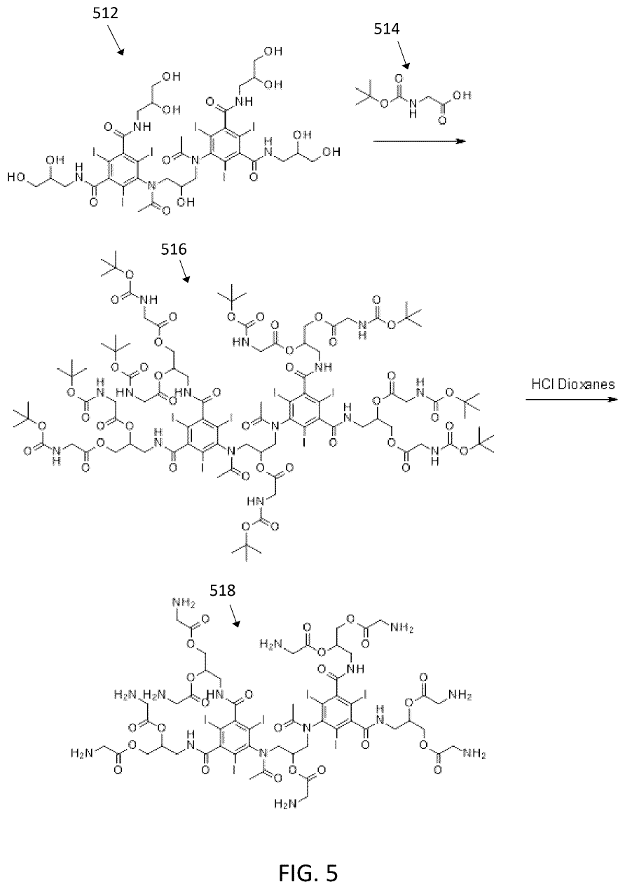

An analogous reaction in which an iodinated polyol is employed is shown in FIG. 5, where iodixanol (512) is allowed to react with an excess of Boc-glycine (514) in the presence of carbodiimide coupling agent such as DCC, to form a Boc-protected iodinated intermediate (516), which is then treated with a deprotecting agent such as HCl in dioxanes, yielding an iodinated nonylamine-functionalized (418) final product, which is a polyamine compound having 9 appended glycine residues that are bonded to a residue of the glycerol through ester bonds.

In various aspects, the present disclosure also provides amino-alcohol-based polyamine compounds that are formed by (a) ester coupling reaction of amine-protected amino alcohols with polycarboxylic acid compounds having two or more carboxyl groups.

Amino-alcohol-based polyamine compounds in accordance with the present disclosure comprise a polycarboxylic acid residue and at least two amino alcohol residues which are each covalently linked to the polycarboxylic acid residue through an ester group.

By performing the ester coupling reaction with a molar excess of amine-protected amino alcohols relative to the total number of moles of carboxyl groups provided by the polycarboxylic acid in the reaction mixture, a single amine-protected amino alcohol will be reacted with each of the carboxyl groups of the polycarboxylic acid, and no carboxyl groups will remain in the resulting amino-alcohol-based polyamine compound. On the other hand, by performing the reaction with a molar excess of carboxyl groups relative to amine-protected amino alcohols, some of the carboxyl groups of the of the polycarboxylic acid will remain unreacted in the resulting amino-alcohol-based polyamine compound.

The ester coupling between the carboxylic acid and hydroxyl groups may be performed in the presence of a suitable coupling agent, for example, a carbodiimide coupling agent, such as N,N′-dicyclohexylcarbodiimide (DCC), 1-Ethyl-3-(3-dimethylaminopropyl) carbodiimide (EDC), or N,N′-Diisopropylcarbodiimide (DIC), among others. The ester linkage that is formed is hydrolysable.

Protective groups for the amine-protected amino alcohols include tert-butoxycarbonyl (Boc) groups, 9-fluorenylmethoxycarbonyl (Fmoc) groups, carboxybenzyl (Cbz) groups, tert-butyl (t-Bu) groups, and benzyl (Bn) groups.

After performing the ester coupling process, the resulting amine-protected amino alcohol resides can be deprotected using known techniques.

Amino alcohols for use in the present disclosure include amino alcohols (e.g., C₁-amino alcohols, C₂-amino alcohols, C₃-amino alcohols, C₄-amino alcohols, C₅-amino alcohols, C₆-amino alcohols, C₇-amino alcohols, C₈-amino alcohols, C₉-amino alcohols, C₁₀-amino alcohols, C₁₁-amino alcohols, C₁₂-amino alcohols, C₁₃-amino alcohols, C₁₄-amino alcohols, C₁₅-amino alcohols, C₁₆-amino alcohols, C₁₇-amino alcohols, C₁₈-amino alcohols, C₁₉-amino alcohols, C₂₀-amino alcohols, etc.) having one hydroxyl group, and one, two, three, four, five, six, seven, eight or more amine groups. Various C₂-C₈-amino alcohols having one, two or three amino groups are shown in the following table.

| | | Amino |
|---|---|---|
| Compound | CAS | functionality |
| Ethanolamine | 141-43-5 | 1 |
| D-Alaninol | 35320-23-1 | 1 |
| (+)-Valinol | 2026-48-4 | 1 |
| trans-4-Aminocyclohexanol | 27489-62-9 | 1 |
| 6-Amino-1-hexanol | 4048-33-3 | 1 |
| 4-Aminophenol | 123-30-8 | 1 |
| 4-Amino-1-butanol | 13325-10-5 | 1 |
| 2-Aminocyclopentanol | 89381-13-5 | 1 |
| 1-Amino-3-(2-aminoethoxy)-2-propanol | 79432-66-9 | 2 |
| 2,2-Bis(2-aminoethoxy)ethanol | 512797-30-7 | 2 |
| 2-[Bis(2-aminoethyl)amino]ethanol | 58145-14-5 | 2 |
| 5-Amino-2-(3-aminopropyl)-1-pentanol | 1392225-96-5 | 2 |
| 1-Amino-3-[2-amino-1- | 98279-42-6 | 3 |
| (aminomethyl)ethoxy]-2-propanol | | |

Regardless of the particular amino alcohol that is selected, any amino alcohol residue that is appended to the polycarboxylic acid will have at least one free amine group at the end of the deprotection process.

Polycarboxylic acids for use in accordance with the present disclosure include polycarboxylic acids having two or more carboxylic acid groups, for example, containing anywhere from 2 to 100 carboxylic acid groups (e.g., having 2 to 3 to 4 to 5 to 6 to 7 to 8 to 9 to 10 to 12 to 15 to 20 to 25 to 30 to 40 to 50 to 60 to 70 to 80 to 90 to 100 carboxylic acid groups).

Illustrative polycarboxylic acids may be selected, for example, from non-iodinated polycarboxylic acids having two or more carboxylic acid groups, including dicarboxylic acids such as C₁-C₈ alkane dicarboxylic acids, including alpha, omega-C₂-C₈ alkane dicarboxylic acids, such as 1,2-ethane dicarboxylic acid, 1,3-propane dicarboxylic acid, 1,4-butane dicarboxylic acid, 1,5-pentane dicarboxylic acid, 1,6-hexane dicarboxylic acid, etc., tricarboxylic acids such as propane-1,2,3-tricarboxylic acid, benzene-1,3,5-tricarboxylic acid, tetracarboxylic acids, pentacarboxylic acids, hexacarboxylic acids, heptacarboxylic acids, octacarboxylic acids, and so forth. Non-iodinated polycarboxylic acids having two or more carboxylic acid groups can also be formed from any of the above-described non-iodinated polyols having two or more hydroxyl group by reacting the hydroxyl groups of the polyols with a cyclic anhydride to form carboxylic-acid groups.

Further illustrative polycarboxylic acids include iodinated polycarboxylic acids. Iodinated polycarboxylic acids include iodinated aromatic polycarboxylic acids, examples of which are compounds that comprise two or more carboxylic acid groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with two or more carboxylic acid groups, which may be directly substituted to the aromatic groups or may be provided in the form of carboxyalkyl groups (e.g., C₁-C₄-carboxyalkyl groups containing one, two, three or four carbon atoms and containing one, two, three or four or more carboxylic acid groups). The carboxyalkyl groups may be linked to the aromatic group directly or through any suitable linking moiety, which may be selected, for example, from amide groups, amine groups, ether groups, ester groups, or carbonate groups, among others.

Iodinated polycarboxylic acids having two or more carboxylic acid groups can be formed from any of the above-described iodinated polyols having two or more hydroxyl group by reacting the hydroxyl groups of the polyols with a cyclic anhydride to form carboxylic-acid groups.

Various C₂-C₅₀-polycarboxylic acids having from two to eight carboxyl groups are shown in the following table.

| | | Functionality |
|---|---|---|
| Compound | CAS# | (q) |
| Succinic Acid | 110-15-6 | 2 |
| Glutaric Acid | 124-04-9 | 2 |
| Adipic Acid | 110-94-1 | 2 |
| Phthalic acid | 88-99-3 | 2 |
| 2,5-Furandicarboxylic acid | 3238-40-2 | 2 |
| 2,4,6-Triiodo-5-(methylamino)-1,3- | 40976-89-4 | 2 |
| benzenedicarboxylic acid | | |
| 2,4,5,6-Tetraiodo-1,3- | 162321-54-2 | 2 |
| benzenedicarboxylic acid | | |
| 1,3,5-benzenetricarboxylic acid | 554-95-0 | 3 |
| 1,2,3-propanetricarboxylic acid | 99-14-9 | 3 |
| 1,2,4-buranetricarboxylic acid | 923-42-2 | 3 |
| 1,3,6-hexanetricarboxylic acid | 1572-40-3 | 3 |
| Citric Acid | 77-92-9 | 3 |
| 2,4,6-Triiodo-1,3,5- | 79211-41-9 | 3 |
| benzenetricarboxylic acid | | |
| 1,2,3,4-Butanetetracarboxylic acid | 1703-58-8 | 4 |
| 1,2,3,4-Cyclobutanetetracarboxylic acid | 53159-92-5 | 4 |
| Iminodisuccinic acid | 7408-20-0 | 4 |
| D-Glutamic acid | 90245-73-1 | 4 |
| 1,2,4,6-Hexanetetracarboxylic acid | 103440-39-7 | 4 |
| Ethylenediaminetetraacetic acid | 60-00-4 | 4 |
| 1,2,4,5-Cyclohexanetetracarboxylic acid | 15383-49-0 | 4 |
| Diethylenetriaminepentaacetic acid | 67-43-6 | 5 |
| Benzenepentacarboxylic acid | 1585-40-6 | 5 |
| Mellitic acid | 517-60-2 | 6 |
| Triethylenetetraminehexaacetic acid | 869-52-3 | 6 |
| 1,2,3,4,5,6- | 2216-84-4 | 6 |
| Cyclohexanehexacarboxylic acid | | |
| 1,3-Benzenedicarboxylic acid, | 1330041-38-7 | 8 |
| 5,5′,5″,5′″-[1,2,4,5- | | |
| benzenetetrayltetrakis | | |
| (methyleneoxy)]tetrakis | | |

It is noted that employing amine-protected amino alcohols that contain two protected amine groups (e.g., 1-amino-3-(2-aminoethoxy)-2-propanol, 2,2-bis(2-aminoethoxy)ethanol, 2-[bis(2-aminoethyl)amino]ethanol, 5-amino-2-(3-aminopropyl)-1-pentanol, etc.) effectively double the number of amine functional groups that can be appended to any given molecule. For instance, molecules such as those shown above that have a q=2 will be able to have four amine functional groups appended, that have a q=3 will be able to have six amine functional groups appended, that have a q=4 will be able to have eight amine functional groups appended, that have a q=5 will be able to have ten amine functional groups appended, that have a q=6 will be able to have twelve amine functional groups appended, and so forth.

Similarly, employing amine-protected amino alcohols that contain three protected amine groups (e.g., 1-amino-3-[2-amino-1-(aminomethyl)ethoxy]-2-propanol, etc.) effectively triple the number of amine functional groups that can be appended to any given molecule. For instance, molecules such as those shown above that have a q=2 will be able to have six amine functional groups appended, that have a q=3 will be able to have nine amine functional groups appended, that have a q=4 will be able to have twelve amine functional groups appended, that have a q=5 will be able to have fifteen amine functional groups appended, that have a q=6 will be able to have eighteen amine functional groups appended, and so forth.

Various specific ester-coupling embodiments will now be discussed.

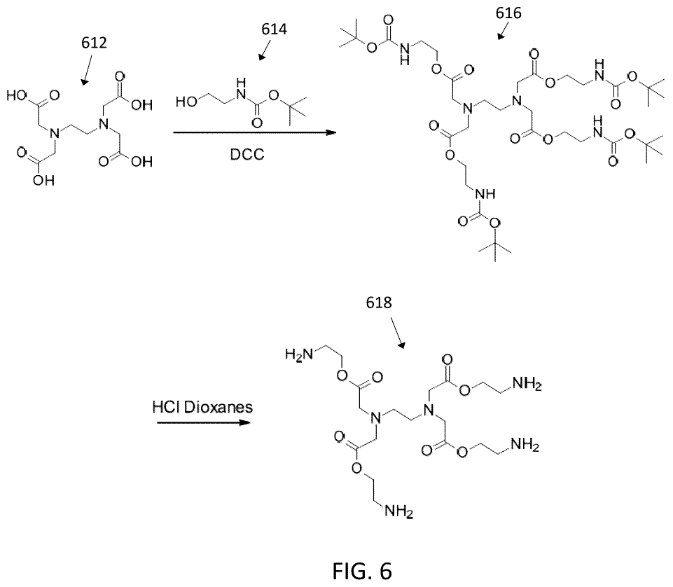

A reaction between a polycarboxylic acid and a protected amino-alcohol is shown in FIG. 6, which schematically illustrates a process in which N,N,N′,N′-tetrakis(carboxymethyl)-ethane-1,2-diamine (612) is allowed to react with an excess of N-Boc-ethanolamine (614) in the presence of a carbodiimide coupling agent such as DCC, to form a Boc-protected intermediate (616), which is then treated with a deprotecting agent such as HCl in dioxane, yielding a tetraamine-functionalized final product (618), which is a polyamine compound having 4 appended ethanolamine residues that are bonded to a residue of the N,N,N′,N′-tetrakis(carboxymethyl)-ethane-1,2-diamine through ester bonds.

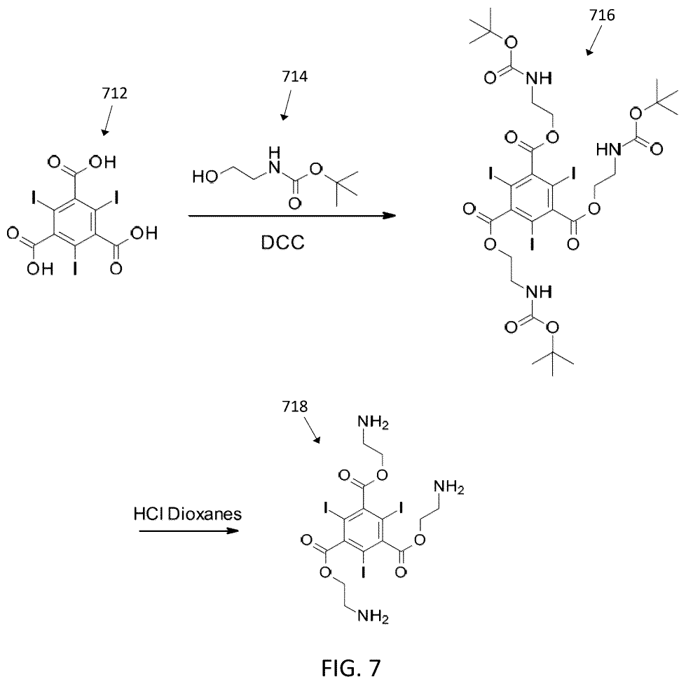

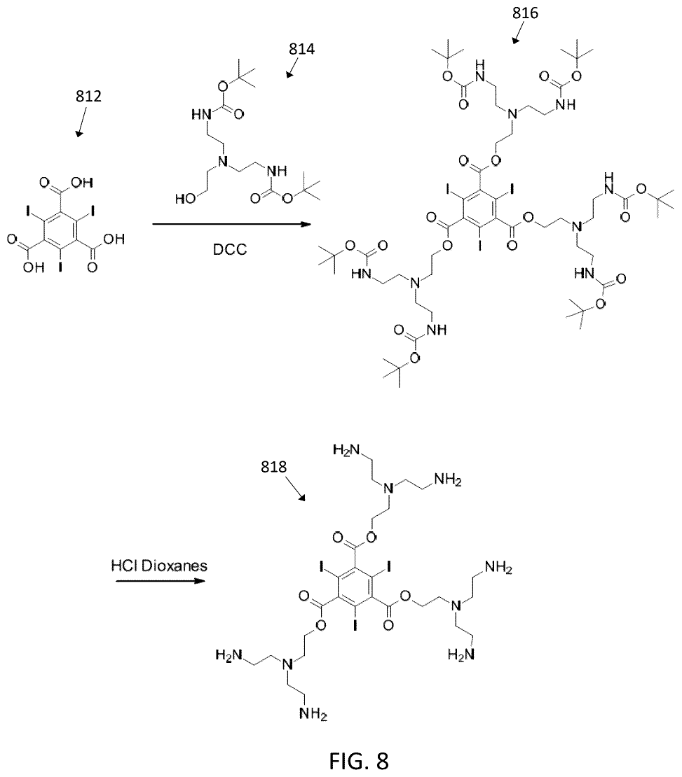

A reaction between an iodinated polycarboxylic acid and a protected amino-alcohol is shown in FIG. 7, which schematically illustrates a process in which 2,4,6-triiodobenzene-1,3,5-tricarboxylic acid (712) is allowed to react with an excess of N-Boc-ethanolamine (714) in the presence of a carbodiimide coupling agent such as DCC, to form a Boc-protected iodinated intermediate (716), which is then treated with a deprotecting agent such as HCl in dioxane, yielding an iodinated triamine-functionalized final product (718), which is a polyamine compound having 3 appended ethanolamine residues that are bonded to a residue of the 2,4,6-triiodobenzene-1,3,5-tricarboxylic acid through ester bonds.

A reaction between an iodinated polycarboxylic acid and a protected amino-alcohol having multiple amino groups is shown in FIG. 8, which schematically illustrates a process in which 2,4,6-triiodobenzene-1,3,5-tricarboxylic acid (812) is allowed to react with an excess of Boc-protected 2-[bis(2-aminoethyl)amino]ethanol (814) in the presence of a carbodiimide coupling agent such as DCC, to form a Boc-protected iodinated intermediate (816), which is then treated with a deprotecting agent such as HCl in dioxane, yielding an iodinated hexylamine-functionalized final product (818), which is a polyamine compound having 3 appended 2-[bis(2-aminoethyl)amino]ethanol residues, each having two amino groups, that are bonded to a residue of the 2,4,6-triiodobenzene-1,3,5-tricarboxylic acid through ester bonds.

Amino-acid- and amino-alcohol-based polyamine compounds in accordance with the present disclosure may be used in various medical applications. In some embodiments, the amino-acid- and amino-alcohol-based polyamine compounds may be used as crosslinking agents, for example, as covalent crosslinking agents, ionic crosslinking agents, or hydrogen-bonding-based crosslinking agents.

In this regard, crosslinked hydrogels are provided in various aspects of the present disclosure that comprise a crosslinked reaction product of (a) an amino-acid- or amino-alcohol-based polyamine compound such as one of those described above and (b) a polymer that forms crosslinks with the amino-acid- or amino-alcohol-based polyamine compound. Such crosslinked hydrogels may be formed in vivo (e.g., using a delivery device like that described below), or such crosslinked hydrogels may be formed ex vivo and subsequently administered to a subject. Such crosslinked hydrogels can be used in a wide variety of biomedical applications, including medical devices, implants, and pharmaceutical compositions.

In embodiments where radiopacity is introduced into the amino-acid- and amino-alcohol-based polyamine compounds of the present disclosure, the crosslinked hydrogels are visible under fluoroscopy. In various embodiments, such crosslinked hydrogels have a radiopacity that is 100 Hounsfield units (HU) or more, beneficially anywhere ranging from 100 HU to 250 HU to 500 HU to 750 HU to 1000 HU to 2500 HU or more.

In some embodiments, the polymer that forms crosslinks with the amino-acid- or amino-alcohol-based polyamine compound is a reactive polymer that comprises a plurality of reactive groups that form covalent crosslinks with the primary amine groups of the amino-acid- or amino-alcohol-based polyamine compound.

Examples of reactive polymers that comprise a plurality of reactive groups that form covalent crosslinks with the primary amine groups of the amino-acid- or amino-alcohol-based polyamine compound include multi-arm polymers that comprise a plurality of polymer arms (e.g., having two, three, four, five, six, seven, eight, nine, ten, eleven, twelve or more arms), wherein two or more polymer arms of the multi-arm polymer comprise one or more reactive end groups. In some embodiments, compositions containing the reactive multi-arm polymers may be provided in which a percentage of the polymer arms comprising one or more reactive end groups may correspond to between 50% and 100% of the total number of polymer arms in the composition (e.g., ranging anywhere from 50% to 70% to 80% to 90% to 95% to 99% to 100% of the total number of polymer arms).

In various embodiments, the polymer arms of the multi-arm polymers are hydrophilic polymer arms, each comprising a comprising a hydrophilic polymer segment.

Hydrophilic polymer segments for the polymer arms can be selected from a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain between 10 and 1000 monomer units.

In some embodiments, the polymer arms of the reactive multi-arm polymers extend from a core region. In certain of these embodiments, the core region comprises a residue of a polyol that is used to form the polymer arms. Illustrative polyols may be selected, for example, from the polyols described above for forming the amino-acid-based polyamine compounds of the present disclosure, among others.

In certain embodiments, the reactive end groups are electrophilic groups. Electrophilic groups may be selected, for example, from cyclic imide ester groups, such as succinimide ester groups,

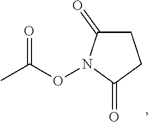

maleimide ester groups, glutarimide ester groups, diglycolimide ester groups, phthalimide ester groups, and bicyclo[2.2.1]hept-5-ene-2,3-dicarboxylic acid imide ester groups,

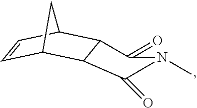

imidazole ester groups, imidazole carboxylate groups, and benzotriazole ester groups, among other possibilities.

The electrophilic groups may be linked to the hydrophilic polymer segment through any suitable linking moiety, which may be selected, for example, from a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups, among others.

In certain embodiments, the linking moiety comprises a hydrolysable ester group. Hydrolysable ester groups may be selected, for example, from glutarate ester groups, succinate ester groups, carbonate ester groups, or adipate ester groups. In particular embodiments, the polymer arms may be terminated with the following reactive, hydrolysable groups, among others: succinimidyl glutarate groups, succinimidyl succinate groups, succinimidyl carbonate groups, or succinimidyl adipate groups.

In other embodiments, the reactive multi-arm polymer does not include a hydrolysable ester group. In this regard, it is noted that the amino-acid- and amino-alcohol-based polyamine compounds provided herein have a hydrolysable ester group.

Further examples of reactive multi-arm polymers are described, for example, in U.S. Patent Application Nos. 2011/0142936, 2021/0061950, 2021/0061954 and 2021/0061957.

In a particular example, a reactive multi-arm polymer like that described above, which comprises a core region and a plurality of hydrophilic polyethylene oxide arms, specifically eight arms) having reactive end groups (e.g., succinimidyl groups) may be covalently crosslinked with an amino-acid- or amino-alcohol-based polyamine compound like that described above, which comprises primary amine groups that are reactive with the reactive end groups of the reactive multi-arm polymer. By reacting the amino-acid- or amino-alcohol-based polyamine compound with the reactive multi-arm polymer under basic conditions, a crosslinked product is formed.

In some embodiments, the polymer that forms crosslinks with the amino-acid- or amino-alcohol-based polyamine compound may be a poly(carboxylic acid) that forms covalent crosslinks with the amino-acid- or amino-alcohol-based polyamine compound, such as poly(acrylic acid), poly(methacrylic acid), alginic acid, hyaluronic acid, pectin, agaropectin, carrageenan, gellan gum, gum arabic, guar gum, xanthan gum, or carboxymethyl cellulose. These and other poly(carboxylic acids) have carboxylic acid groups that can form amide bonds with primary amine groups of the amino-acid- or amino-alcohol-based polyamine compound. Such crosslinks may be formed, for example, in the presence of a carbodiimide coupling reagent. In a particular example, hyaluronic acid may be covalently crosslinked with an amino-acid- or amino-alcohol-based polyamine compound as described herein, which comprises primary amine groups that form amide bonds with the carboxylic acid groups of the hyaluronic acid, thereby forming a crosslinked product.

In some embodiments, the polymer that forms crosslinks with the amino-acid- or amino-alcohol-based polyamine compound is an anionic polymer that comprises a plurality of anionic groups that can ionically crosslink with cationic primary amine groups of the amino-acid- or amino-alcohol-based polyamine compound. In some embodiments, the anionic polymer is water soluble.

Examples of anionic polymers that comprise a plurality of anionic groups that can ionically crosslink with cationic primary amine groups of the amino-acid- or amino-alcohol-based polyamine compound include any of a variety of synthetic, natural, or hybrid synthetic-natural anionic polymers that comprise one or more groups selected from carboxylate groups, sulfonate groups, sulfate groups, phosphate groups, or phosphonate groups, which are negatively charged at a pH greater than or equal to 7 or more and in some cases are negatively charged at a pH greater than 6, greater than 4, or even greater than 2. Particular examples of anionic polymers include poly(carboxylic acids) such as poly(acrylic acid) or poly(methacrylic acid), anionic polysaccharides including alginates, hyaluronates, pectins, agaropectins, carrageenans, gellan gum, gum arabic, guar gum, xanthan gum, and carboxymethyl celluloses, sulfonate polymers such as poly(2-acrylamido-2-methylpropane sulfonate) (polyAMPS) or polystyrene sulfonate, and polyphosphates. The anionic polymer may be provided in a salt form, for example, in a sodium salt form or a potassium salt form, among others.

In some aspects of the present disclosure, systems are provided that are configured to dispense an amino-acid- or amino-alcohol-based polyamine compound and a polymer that forms crosslinks with the amino-acid- or amino-alcohol-based polyamine compound under conditions such that the amino-acid- or amino-alcohol-based polyamine compound and the polymer crosslink with one another. Such systems can be used to form crosslinked hydrogels, either in vivo or ex vivo.

In some embodiments, systems are provided that are configured to dispense an amino-acid- or amino-alcohol-based polyamine compound and a reactive polymer that comprises a plurality of reactive groups that form covalent crosslinks with the primary amine groups of the amino-acid- or amino-alcohol-based polyamine compound under conditions such that the amino-acid- or amino-alcohol-based polyamine compound and the reactive polymer covalently crosslink with one another. Exemplary amino-acid- and amino-alcohol-based polyamine compounds and exemplary reactive polymers are described above, which reactive polymers include reactive multi-arm polymers that comprise a plurality of polymer arms, wherein two or more polymer arms of the multi-arm polymer comprise one or more reactive end groups, such as cyclic imide ester groups. In certain embodiments, those conditions comprise an environment having a basic pH, for example, a pH ranging from about 9 to about 11.

In some embodiments, a system is provided that comprises (a) a first composition that comprises an amino-acid- or amino-alcohol-based polyamine compound and (b) a second composition that comprises a reactive polymer that forms covalent crosslinks with the amino-acid- or amino-alcohol-based polyamine compound. In some embodiments, a third composition in the form of an accelerant composition is also provided.

The first composition may be a first fluid composition comprising the amino-acid- or amino-alcohol-based polyamine compound or a first dry composition that comprises the amino-acid- or amino-alcohol-based polyamine compound, to which a suitable fluid such as water for injection, saline, etc. can be added to form a first fluid composition. In addition to the amino-acid- or amino-alcohol-based polyamine compound, the first composition may further comprise additional agents, including those described below. The first composition may be provided in a suitable reservoir such as a syringe, vial or other reservoir.

The second composition may be a second fluid composition comprising the reactive multi-arm polymer or a second dry composition that comprises the reactive multi-arm polymer, to which a suitable fluid such as water for injection, saline, etc. can be added to form a second fluid composition. In addition to the reactive multi-arm polymer, the second composition may further comprise additional agents, including those described below. The second composition may be provided in a suitable reservoir such as a syringe, vial or other reservoir.

The accelerant composition may be a fluid accelerant composition that is buffered to a basic pH or a dry composition that comprises a basic buffering composition to which a suitable fluid such as water for injection, saline, etc. can be added to form a fluid accelerant composition that is buffered to a basic pH. The accelerant composition may further comprise additional agents, including those described below. The accelerant composition may be provided in a suitable reservoir such as a syringe, vial or other reservoir.

In some embodiments, the amino-acid- or amino-alcohol-based polyamine compound is initially combined with the reactive multi-arm polymer at an acidic pH at which covalent crosslinking between the reactive groups of the reactive multi-arm polymer and the primary amine groups of the amino-acid- or amino-alcohol-based polyamine compound is suppressed. Then, when covalent crosslinking is desired, a pH of the mixture of the amino-acid- or amino-alcohol-based polyamine compound and the reactive multi-arm polymer is changed from an acidic pH to a basic pH, leading to covalent crosslinking between the same.

In some embodiments, a system is provided wherein a precursor fluid composition is prepared, which comprises the amino-acid- or amino-alcohol-based polyamine compound and the reactive multi-arm polymer and is buffered to an acidic pH. For example, the precursor fluid composition may have a pH ranging, for example, from about 3 to about 5, among other possibilities. In some embodiments, the precursor fluid composition contains an acidic buffer that comprise monobasic sodium phosphate, among other possibilities. The precursor fluid composition may further comprise additional agents, including those described below.

The system also includes a fluid accelerant composition that comprises a basic buffer. For example, the fluid accelerant may contain a basic buffer that provides the fluid accelerant with a pH ranging, for example, from about 9 to about 11. In some embodiments, the accelerator composition may comprise sodium borate and dibasic sodium phosphate, among other possibilities. The fluid accelerant composition may further comprise additional agents, including those described below.

The precursor fluid composition that is buffered to an acidic pH and comprises the amino-acid- or amino-alcohol-based polyamine compound and the reactive multi-arm polymer and the fluid accelerant composition that is buffered to basic pH may be combined form covalently crosslinked hydrogels, either in vivo or ex vivo.

In other embodiments, systems are provided that are configured to dispense an amino-acid- or amino-alcohol-based polyamine compound as described herein and an anionic polymer that comprises a plurality of anionic groups that ionically crosslink with cationic primary amine groups of the amino-acid- or amino-alcohol-based polyamine compound under pH conditions such that the amino-acid- or amino-alcohol-based polyamine compound and the anionic polymer ionically crosslink with one another. Exemplary amino-acid- and amino-alcohol-based polyamine compounds and anionic polymers are described above.

In some embodiments, a system is provided that comprises (a) a first composition that comprises an amino-acid- or amino-alcohol-based polyamine compound and (b) a second composition that comprises an anionic polymer that forms ionic crosslinks with the amino-acid- or amino-alcohol-based polyamine compound. In some embodiments, a third composition in the form of an accelerant composition is provided.

The first composition may be a first fluid composition comprising the amino-acid- or amino-alcohol-based polyamine compound or a first dry composition that comprises the amino-acid- or amino-alcohol-based polyamine compound, to which a suitable fluid such as water for injection, saline, etc. can be added to form a first fluid composition. In addition to the amino-acid- or amino-alcohol-based polyamine compound, the first composition may further comprise additional agents, including those described below. The first composition may be provided in a suitable reservoir such as a syringe, vial or other reservoir.

The second composition may be a second fluid composition comprising the anionic polymer or a second dry composition that comprises the anionic polymer, to which a suitable fluid such as water for injection, saline, etc. can be added to form a second fluid composition. In addition to the anionic polymer, the second composition may further comprise additional agents, including those described below. The second composition may be provided in a suitable reservoir such as a syringe, vial or other reservoir.

The accelerant composition may be a fluid accelerant composition that is buffered to a basic or acidic pH or a dry composition that comprise a basic or acidic buffering composition to which a suitable fluid such as water for injection, saline, etc. can be added to form a fluid accelerant composition that is buffered to a basic or acidic pH. The accelerant composition may further comprise additional agents, including those described below. The accelerant composition may be provided in a suitable reservoir such as a syringe, vial or other reservoir.

In some embodiments, the amino-acid- or amino-alcohol-based polyamine compound is initially combined with the anionic polymer either at a basic pH at which the primary amine groups of the amino-acid- or amino-alcohol-based polyamine compound are neutrally charged, or at an acidic pH at which the carboxyl groups of the anionic are neutrally charged, such that ionic crosslinking is suppressed. Then, when crosslinking is desired, a pH of the mixture of the amino-acid- or amino-alcohol-based polyamine compound and the anionic polymer is changed from an acidic or basic pH to a more neutral pH, leading to ionic crosslinking between the amino-acid- or amino-alcohol-based polyamine compound and the anionic polymer.

In some embodiments, a system is provided wherein a precursor fluid composition is prepared that is buffered to an acidic or basic pH and comprises the amino-acid- or amino-alcohol-based polyamine compound and the anionic polymer. The precursor fluid composition may further comprise additional agents, including those described below.

The system also includes a fluid accelerant composition that is buffered to a basic or acidic pH. The amount and type of buffering agent in the fluid accelerant composition are selected such that when combined with the precursor fluid composition that contains the amino-acid- or amino-alcohol-based polyamine compound and the anionic polymer, the resulting mixture has a more neutral pH at which the amino-acid- or amino-alcohol-based polyamine compound is positively charged and the anionic polymer is negatively charged, such that ionic crosslinks form between the same. The fluid accelerant composition may further comprise additional agents, such as those descried below.

The precursor fluid composition that is buffered to an acidic or basic pH and comprises the amino-acid- or amino-alcohol-based polyamine compound and the anionic polymer and the fluid accelerant composition that is buffered to a basic or acidic pH may be combined form ionically crosslinked hydrogels, either in vivo or ex vivo.

Examples of additional agents for use in the above-described compositions include therapeutic agents such anti-angiogenic agents, cytotoxic agents, chemotherapeutic agents, checkpoint inhibitors, immune modulatory cytokines, T-cell agonists, and STING (stimulator of interferon genes) agonists.

Examples of additional agents further include imaging agents in addition to any iodine that may be present in the radiopaque products. Such imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) radiocontrast agents, such as those based on the clinically important isotope 99mTc, as well as other gamma emitters such as 123I, 125I, 131I, 111In, 57Co, 153Sm, 133Xe, 51Cr, 81mKr, 201Tl, 67Ga, and 75Se, among others, (e) positron emitters, such as 18F, 11C, 13N, 15O, and 68Ga, among others, may be employed to yield functionalized radiotracer coatings, (f) radiocontrast agents, for example, particles of tantalum, tungsten, rhenium, niobium, molybdenum, and their alloys, which metallic particles may be spherical or non-spherical, non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®), and (g) contrast agents for use in connection with near-infrared (NIR) imaging, which can be selected to impart near-infrared fluorescence to the coatings of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others. NIR-sensitive dyes include cyanine dyes, squaraines, phthalocyanines, porphyrin derivatives and borondipyrromethane (BODIPY) analogs, among others.

In various embodiments, systems are provided that include one or more delivery devices for delivering the compositions described herein to a subject.

In some embodiments, a delivery device is provided that includes (a) a first reservoir that contains a first fluid composition that comprises an amino-acid- or amino-alcohol-based polyamine compound as described above and (b) a second reservoir that contains a second fluid composition that comprises a reactive multi-arm polymer as described above.

In some embodiments, a delivery device is provided that includes (a) a first reservoir that contains a first fluid composition that comprises an amino-acid- or amino-alcohol-based polyamine compound as described above and a reactive multi-arm polymer as described above, wherein the first fluid composition is buffered to an acidic pH to inhibit covalent crosslinking, such as the precursor fluid composition previously described and (b) a second reservoir that contains a second fluid composition, such as the fluid accelerant composition described above.

In some embodiments, a delivery device is provided that includes (a) a first reservoir that contains a first fluid composition that comprises an amino-acid- or amino-alcohol-based polyamine compound as described above and (b) a second reservoir that contains a second fluid composition that comprises an anionic polymer as described above.

In some embodiments, a delivery device is provided that includes (a) a first reservoir that contains a first fluid composition that comprises an amino-acid- or amino-alcohol-based polyamine compound as described above and an anionic polymer as described above, the first fluid composition being buffered to either an acidic or a basic pH to inhibit ionic crosslinking, such as the precursor fluid composition previously described and (b) a second reservoir that contains a second fluid composition, such as the fluid accelerant composition that is buffered to a basic or acidic pH, as described above.

In each of the above cases, during operation, the first composition and second composition are dispensed from the first and second reservoirs and combined, whereupon the amino-acid- or amino-alcohol-based polyamine compound and the reactive multi-arm polymer and crosslink with one another to form a hydrogel.

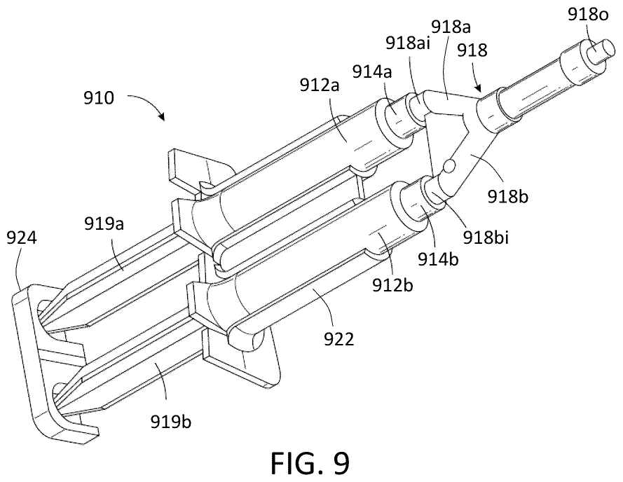

In particular embodiments, and with reference to FIG. 9, the system may include a delivery device 910 that comprises a double-barrel syringe, which includes a first barrel 912a having a first barrel outlet 914a, which first barrel contains the first fluid composition, a first plunger 916a that is movable in the first barrel 912a, a second barrel 912b having a second barrel outlet 914b, which second barrel 912b contains the second fluid composition, and a second plunger 916b that is movable in the second barrel 912b. In some embodiments, the device 910 may further comprise a mixing section 918 having a first mixing section inlet 918ai in fluid communication with the first barrel outlet 914a, a second mixing section inlet 918bi in fluid communication with the second barrel outlet, and a mixing section outlet 9180. Also shown are a syringe holder 922 configured to hold the first and second syringe barrels 912a, 912b, in a fixed relationship and a plunger cap 924 configured to hold the first and second plungers 916a, 916b in a fixed relationship.

In some embodiments, the delivery device may further comprise a cannula or catheter tube that is configured to receive first fluid composition and second fluid composition from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first fluid composition and second fluid composition are dispensed from the first and second barrels, whereupon the first fluid composition and second fluid composition mix and ultimately crosslink to form a crosslinked hydrogel, which is administered onto or into tissue of a subject. For example, the first fluid composition and second fluid composition may pass from the first and second barrels, into the mixing section via first and second mixing section inlets, whereupon the first fluid composition and second fluid composition are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first fluid composition and the second fluid compositions may pass from the first and second lumen into a mixing section at a distal end of the multi-lumen catheter via first and second mixing section inlets, respectively, whereupon the first fluid composition and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique such as ultrasound or an X-ray based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments.

1. An amino-acid-based polyamine compound that is formed by (a) a ring-opening reaction of two or more amino acid N-carboxyanhydride molecules with a polyol having two or more hydroxyl groups under acid catalyzed conditions or (b) an ester coupling reaction of two or more amine-protected amino acid molecules with a polyol having two or more hydroxyl groups in the presence of an ester coupling agent.

2. The amino-acid-based polyamine compound of claim 1, wherein the amino acid N-carboxyanhydride molecules are selected from glycine N-carboxyanhydride, lysine N-carboxyanhydride, ornithine N-carboxyanhydride, and cysteine N-carboxyanhydride.

3. The amino-acid-based polyamine compound of claim 1, wherein the amine-protected amino acid is selected from amine-protected glycine, amine-protected lysine, amine-protected ornithine, and amine-protected cysteine.

4. The amino-acid-based polyamine compound of claim 1, wherein the polyol has between 3 and 20 hydroxyl groups.

5. An amino-acid-based polyamine compound that comprises (a) a residue of a polyol having two or more hydroxyl groups and (b) two or more single amine-terminated amino acid residues that are each linked by an ester bond at a site of a residue of one of the two or more hydroxyl groups.

6. The amino-acid-based polyamine compound of claim 5, wherein the amine-terminated amino acid residues are selected from amine-terminated glycine residues, amine-terminated lysine residues, amine-terminated ornithine residues, and amine-terminated cysteine residues.

7. The amino-acid-based polyamine compound of claim 5, wherein the residue of the polyol is a residue of a polyol has between 3 and 20 hydroxyl groups.

8. An amino-alcohol-based polyamine compound that is formed by an ester coupling reaction of (a) two or more amine-protected amino alcohol molecules having one or more amino groups and a single hydroxyl group with (b) a polycarboxylic acid compound having two or more carboxyl groups in the presence of an ester coupling agent.

9. The amino-alcohol-based polyamine compound of claim 8, wherein the amine-protected amino alcohol is an amine-protected C₂-C₁₀-amino alcohol having one, two, three or four amino groups and a single hydroxyl group.

10. The amino-alcohol-based polyamine compound of claim 8, wherein the polycarboxylic acid has between 3 and 20 carboxyl groups.

11. A system for forming a hydrogel that comprises the amino-acid-based polyamine compound of claim 1 and a polymer that that forms crosslinks with the amino-acid-based polyamine compound.

12. The system of claim 11, wherein the polymer that forms crosslinks with the amino-acid-based polyamine compound is a reactive multi-arm polymer that comprises a plurality of hydrophilic polymer arms comprising hydrophilic polymer segments and reactive end groups that covalently crosslink with primary amine groups of the amino-acid-based polyamine compound.

13. The system of claim 12, wherein the hydrophilic polymer segments are selected from polyalkylene oxide segments, polyester segments, polyoxazoline segments, polydioxanone segments, and polypeptide segments.

14. The system of claim 12, wherein the reactive groups are electrophilic groups selected from imidazole esters, imidazole carboxylates, benzotriazole esters, or imide esters.

15. The system of claim 12, wherein the reactive groups are cyclic imide ester groups.

16. The system of claim 11, wherein the polymer that forms crosslinks with the amino-acid-based polyamine compound is an anionic polymer that comprises a plurality of anionic groups that ionically crosslink with the plurality of primary amine groups of the amino-acid-based polyamine compound.

17. The system of claim 16, wherein the anionic polymer comprises a plurality of carboxyl groups, sulfonate groups, sulfate groups, phosphate groups, or phosphonate groups.

18. A medical hydrogel formed by crosslinking the amino-acid-based polyamine compound and the polymer that forms crosslinks with the amino-acid-based polyamine compound of the system of claim 1.

19. A method of treatment comprising administering to a subject a mixture that comprises the amino-acid-based polyamine compound of claim 1 and a polymer that forms crosslinks with the amino-acid-based polyamine compound under conditions such that the amino-acid-based or amino-alcohol-based polyamine compound and the polymer crosslink after administration.

20. The method of claim 19, wherein the polymer that forms crosslinks with the amino-acid-based polyamine compound is a reactive multi-arm polymer that comprises a plurality of hydrophilic polymer arms comprising hydrophilic polymer segments and reactive end groups that covalently crosslink with primary amine groups of the amino-acid-based polyamine compound.
